(19) United States
(12) Patent Application Publication
(10) Pub. No.: US 2019/0387986 A1
(43) Pub. Date: Dec. 26, 2019

(54) BLOOD PRESSURE MEASUREMENT APPARATUS, METHOD, AND PROGRAM

(71) Applicant: OMRON HEALTHCARE Co., Ltd., Muko-shi (JP)

(72) Inventors: Eriko Kan, Kyoto (JP); Mayumi Akatsuka, Kyoto (JP); Tatsunori Ito, Kyoto (JP)

(73) Assignee: OMRON HEALTHCARE Co., Ltd., Muko-shi (JP)

(21) Appl. No.: 16/561,892

(22) Filed: Sep. 5, 2019

Related U.S. Application Data
(63) Continuation of application No. PCT/JP2018/009577, filed on Mar. 12, 2018.

(30) Foreign Application Priority Data
Mar. 15, 2017 (JP) 2017-050489

(51) Int. Cl. A61B 5/021; A61B 5/00

(52) U.S. Cl. CPC A61B 5/02116; A61B 5/6843; A61B 5/7221

(57) ABSTRACT

A degree of reliability of blood pressure data including a blood pressure value for each heartbeat, which is obtained by measuring blood pressure using one or more pressure sensors, is calculated. A blood pressure measurement apparatus according to an aspect includes: a blood pressure meter configured to obtain blood pressure data including a blood pressure value for each heartbeat by detecting a pressure pulse wave using one or more sensors; an extraction unit configured to extract one or more feature amounts of the blood pressure data; and a calculation unit configured to calculate a degree of reliability indicating how accurately the blood pressure data indicates the blood pressure values, based on the feature amount.

### TECHNICAL FIELD

The present invention relates to a blood pressure measurement apparatus, a method, and a program, for continuously measuring biological information.

### BACKGROUND ART

Accompanying the development of sensor technology, an environment has been created in which high-performance sensors can easily be used, and thus it has become increasingly important in medicine to use biological information to sense an abnormality in a living body at an early stage, which is useful for treatment.

A blood pressure measurement apparatus using tonometry has been known, in which biological information such as a pulse and blood pressure can be measured using information detected by a pressure sensor in a state in which the pressure sensor is directly in contact with part of a living body through which an artery, such as the radial artery of a wrist, passes (e.g., see JP 2004-222847A).

The blood pressure measurement apparatus according to JP 2004-222847A calculates the degree of reliability of blood pressure information by determining whether or not the sensor is in an unsuitable arrangement state with respect to the artery to be subjected to measurement.

### SUMMARY OF INVENTION

However, with the blood pressure measurement apparatus disclosed in JP 2004-222847, although the degree of reliability is calculated based on the contact state at the determination timing, the calibration method is determined based on the arrangement state at the time of calibration, and the blood pressure value is calculated using the calibration method. Therefore, even if the sensor is not in an unsuitable arrangement state, if the current state is different from the contact state at the time of calibration, a correct blood pressure value will not be calculated.

The present invention was made with attention given to the foregoing circumstances, and it is an object thereof to provide a blood pressure measurement apparatus, a method, and a program, according to which it is possible to calculate the degree of reliability of blood pressure data including a blood pressure value per heartbeat, which is obtained by measuring the blood pressure using one or more sensors.

In order to solve the above-described problem, a first aspect of the present invention is a blood pressure measurement apparatus including: a blood pressure meter configured to obtain blood pressure data including a blood pressure value for each heartbeat by detecting a pressure pulse wave using one or more sensors; an extraction unit configured to extract one or more feature amounts of the blood pressure data; and a calculation unit configured to calculate a degree of reliability indicating how accurately the blood pressure data indicates blood pressure values, based on the feature amount.

In a second aspect of the present invention, the extraction unit extracts at least one feature amount among a stability feature amount indicating whether or not the pressure pulse wave is stable, a sensor contact state feature amount indicating whether or not a state of contact between the one or more sensors and a measurement site is normal, and a similarity degree feature amount indicating a degree of similarity between the pressure pulse wave at a measurement start time and the pressure pulse wave at a desired measurement time, and the calculation unit calculates the degree of reliability based on the at least one feature amount.

In a third aspect of the present invention, if it is determined that the pressure pulse wave is stable and it is determined that the state of contact is normal, and furthermore, if it is determined that the degree of similarity is greater than a threshold value, the calculation unit sets the degree of reliability for that segment as being high.

According to the first aspect of the present invention, the blood pressure measurement apparatus obtains blood pressure data including a blood pressure value per heartbeat by detecting a pressure pulse wave using one or more sensors, extracts one or more feature amounts of the blood pressure data, calculates a degree of reliability indicating how accurately the blood pressure data indicates the blood pressure values based on the feature amount, and thereby is able to evaluate the degree of reliability for the measured blood pressure each instance of measurement, and is able to evaluate the degree of reliability for the measured blood pressure in accordance with the measurement subject.

According to the second aspect, the extraction unit extracts at least one feature amount among a stability feature amount indicating whether or not the pressure pulse wave is stable, a sensor contact state feature amount indicating whether or not a state of contact between one or more sensors included in a blood pressure meter and the measurement site is normal, and a similarity degree feature amount indicating the degree of similarity between the pressure pulse wave at the measurement start time and the pressure pulse wave the desired measurement time, and the calculation unit calculates the degree of reliability based on the at least one feature amount, and thereby calculates the degree of reliability based on at least one of whether or not the pressure pulse wave is stable, whether or not the contact state between the sensor and the measurement site is stable, and whether or not the pressure pulse wave at the measurement start time and the pressure pulse wave at the measurement time are similar. Therefore, it is possible to calculate a degree of similarity based on any one of these feature amounts. As a result, it is possible to evaluate the degree of reliability for the measured blood pressure value, which is the degree of reliability unique to one feature amount, for each instance of measurement.

According to the third aspect of the present invention, if it is determined that the stability of the pressure pulse wave is high, the state of contact between the sensor and the contact site is normal, and the degree of similarity is less than or equal to a threshold value, it is understood that the degree of reliability has dropped slightly.

Since it is possible to precisely calculate the degree of reliability of the blood pressure data obtained through measurement and the degree of reliability also changes in some cases due to the measurement time being different in the same body, it is possible to obtain a degree of reliability that matches the measurement status in an instance of measurement. As a result, it is possible to more reliably obtain chronological data of highly-accurate blood pressure values.

That is, according to the aspects of the invention, it is possible to provide a blood pressure measurement apparatus, a method, and a program, according to which it is possible to calculate the degree of reliability of blood pressure data including a blood pressure value per heartbeat, which is obtained by measuring the blood pressure.

### BRIEF DESCRIPTION OF DRAWINGS

FIG. 1 is block diagram showing a blood pressure measurement apparatus according to an embodiment.

FIG. 2 is a block diagram showing a blood pressure meter included in the blood pressure measurement apparatus of FIG. 1.

FIG. 3 is a diagram showing an example in which the blood pressure measurement apparatus of FIG. 1 is attached to a wrist.

FIG. 4 is a cross-sectional view of a wrist to which the blood pressure measurement apparatus of FIG. 3 is attached.

FIG. 5 is a diagram showing an example of an arrangement of sensors of FIGS. 2 to 4.

FIG. 6 is a diagram showing a distribution of AC components of blood pressure values acquired using the sensors of FIGS. 2 to 4.

FIG. 7 is a diagram showing a distribution of DC components of blood pressure values acquired using the sensors of FIGS. 2 to 4.

FIG. 8 is a diagram showing change over time in a pressure pulse wave and blood pressure values per heartbeat therein.

FIG. 9A is a diagram showing an example of feature amounts obtained from a distribution of AC components of a tonogram.

FIG. 9B is a diagram showing an example of feature amounts obtained from a distribution of DC components of a tonogram.

FIG. 9C is a diagram showing another example of feature amounts obtained from a distribution of DC components of a tonogram.

FIG. 10 is a diagram showing an example of segments determined by the measurement stability determination unit of FIG. 1.

FIG. 11 is a diagram showing an example of a tonometry state determined by a sensor contact state determination unit of FIG. 1.

FIG. 12 is a flowchart showing operations of the blood pressure measurement apparatus of FIG. 1.

FIG. 13 is a diagram showing an example of equipping the blood pressure measurement apparatus of FIG. 1.

### DESCRIPTION OF EMBODIMENTS

Hereinafter, a blood pressure measurement apparatus, a method, and a program of an embodiment according to the present invention will be described with reference to the drawings. Note that in the following embodiment, it is assumed that portions denoted by identical numbers perform the same operations, and redundant description thereof will be omitted.

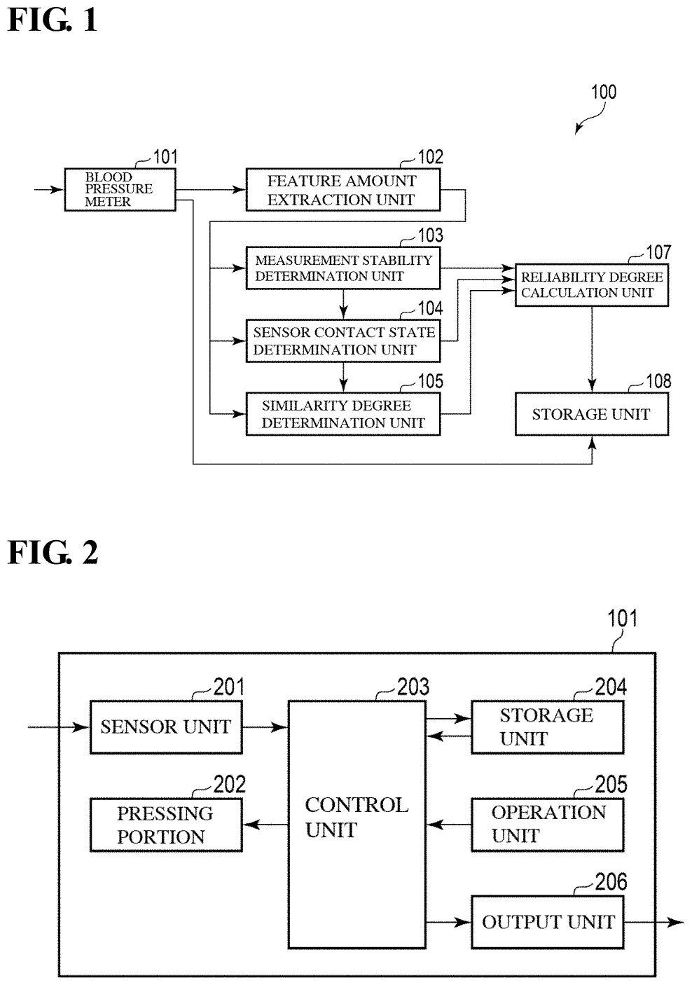

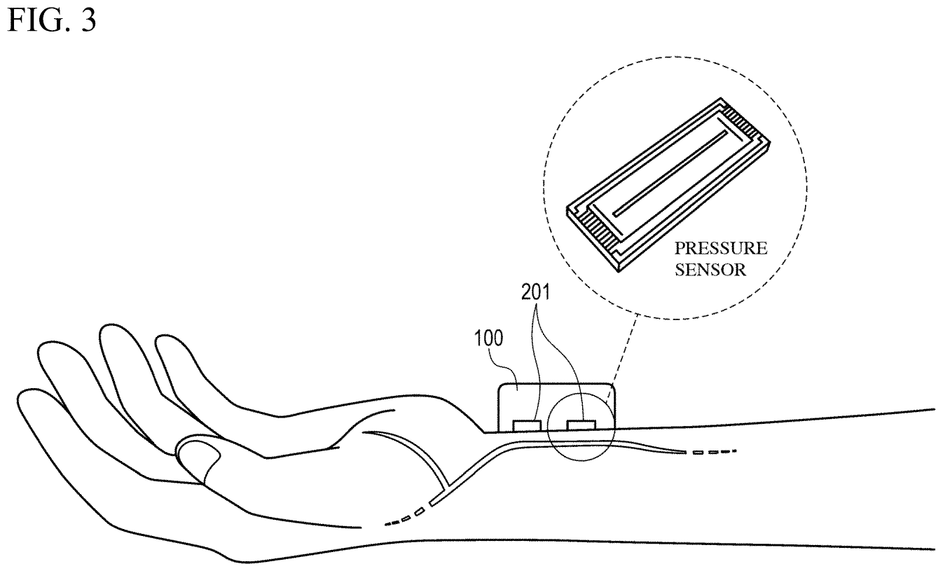

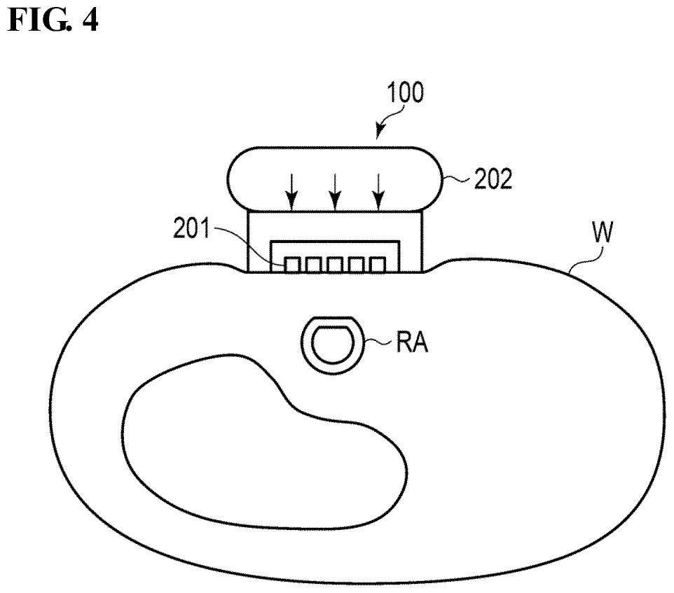

A blood pressure measurement apparatus 100 according to the present embodiment will be described with reference to FIGS. 1 to 5. FIG. 1 is a diagram showing functional blocks of the blood pressure measurement apparatus 100, and shows a blood pressure meter 101 that continuously measures blood pressure over time, a measurement stability determination unit 103, a sensor contact state determination unit 104, a similarity degree determination unit 105, a reliability degree calculation unit 107, and a storage unit 108. FIG. 2 is a diagram showing functional blocks of the blood pressure meter 101, which can continuously measure blood pressure over time for each heartbeat, based on pressure pulse wave information. In the present embodiment, mainly a case of using the blood pressure meter 101, which employs tonometry, will be described. Note that the blood pressure meter 101 is not limited to a blood pressure meter employing tonometry, and it is also possible to use any blood pressure meter that can measure a pressure pulse wave using one or multiple sensors. FIG. 3 is a diagram showing an image in which, as an example, the blood pressure measurement apparatus 100 employing a tonometry scheme is attached, and FIG. 3 is a schematic transparent view from the side of a palm of a hand (viewed from the direction in which the fingers are aligned when the hand is spread out). FIG. 3 shows an example in which pressure sensors are aligned in two rows intersecting the radial artery. In FIG. 3, it appears as if the blood pressure measurement apparatus 100 has merely been placed on the palm side of the arm, but in actuality, the blood pressure measurement apparatus 100 has been wrapped around the arm.

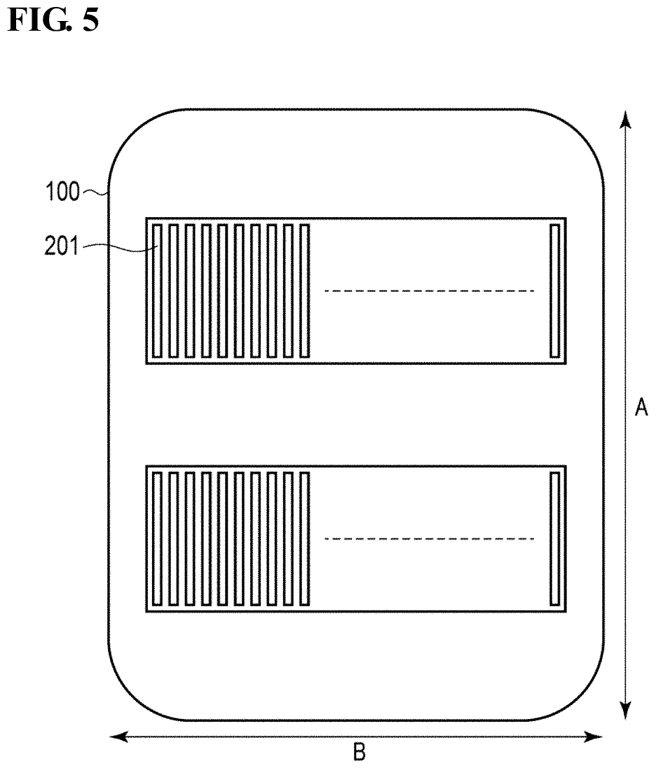

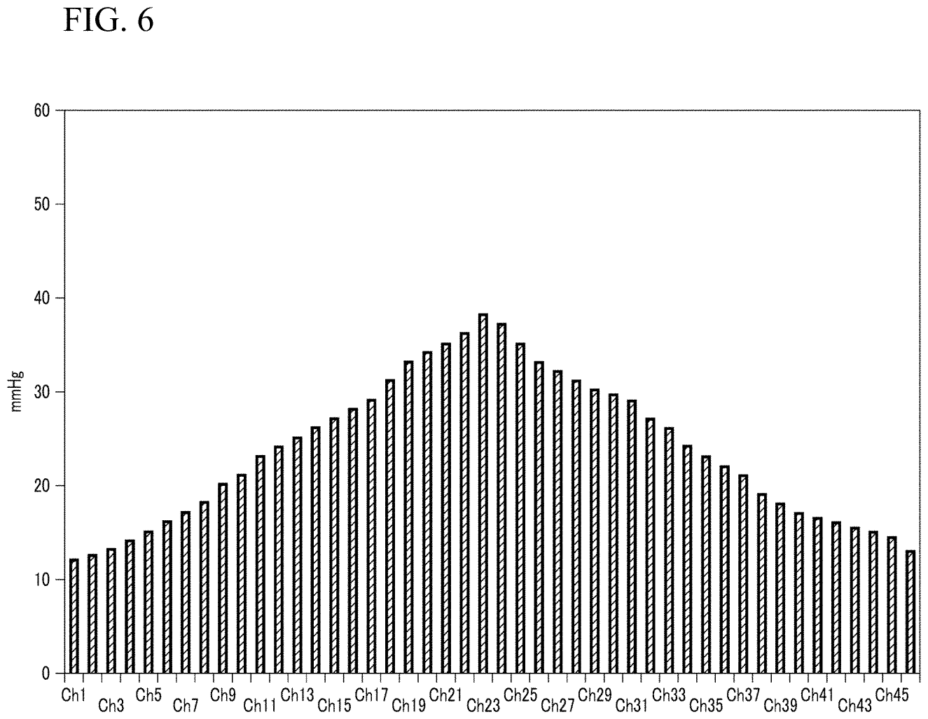

FIG. 4 is a cross-sectional view of the blood pressure measurement apparatus 100 and a wrist W at the position of a sensor unit 201, in a state in which the blood pressure measurement apparatus 100 is attached to the wrist. FIG. 4 also shows that the radial artery RA is being pressed by the blood pressure measurement apparatus 100 and the upper portion of the radial artery RA has been flattened. FIG. 5 is a view from the side of the blood pressure measurement apparatus 100 that comes into contact with a living body, and sensor units 201 are arranged in parallel in two rows on the surface that comes into contact. In the sensor units 201, multiple sensors are aligned in a direction B, which intersects a direction A in which the radial artery extends when the blood pressure measurement apparatus 100 is attached to the wrist W.

As shown in FIG. 1, the blood pressure measurement apparatus 100 includes a blood pressure meter 101, a feature amount extraction unit 102, a measurement stability determination unit 103, a sensor contact state determination unit 104, a similarity degree determination unit 105, a reliability degree calculation unit 107, and a storage unit 108.

For example, the blood pressure measurement apparatus 100 is ring-shaped, wraps like a bracelet around a wrist or the like, and measures blood pressure based on biological information. As shown in FIGS. 2 and 3, the blood pressure measurement apparatus 100 is arranged such that the sensor portions 201 (specifically, the pressure sensors) are located above the radial artery. Also, the blood pressure measurement apparatus 100 is preferably arranged in accordance with the height of the heart.

The blood pressure meter 101 measures the pressure pulse wave per heartbeat, which is chronologically continuous, using tonometry. Tonometry is a method of measuring pressure pulse waves and determining blood pressure by pressing a blood vessel with a pressure sensor. If a blood vessel is regarded as a circular tube with a uniform thickness, a relational expression between the inner pressure of the blood vessel (blood pressure) and the outer pressure of the blood vessel (pressure of the pressure pulse wave) can be derived in accordance with Laplace's law with consideration given to the blood vessel walls, regardless of the flow of blood in the blood vessel and whether or not there is a pulse. With this relational expression, under the condition that the blood vessel has been pressed in a pressing plane, the blood pressure can be approximated as being equal to the pressure of the pressure pulse wave by approximating the radii of the outer wall and the inner wall of the blood vessel. As a result, the blood pressure meter 101 measures the blood pressure value of the living body to which it is attached for each heartbeat.

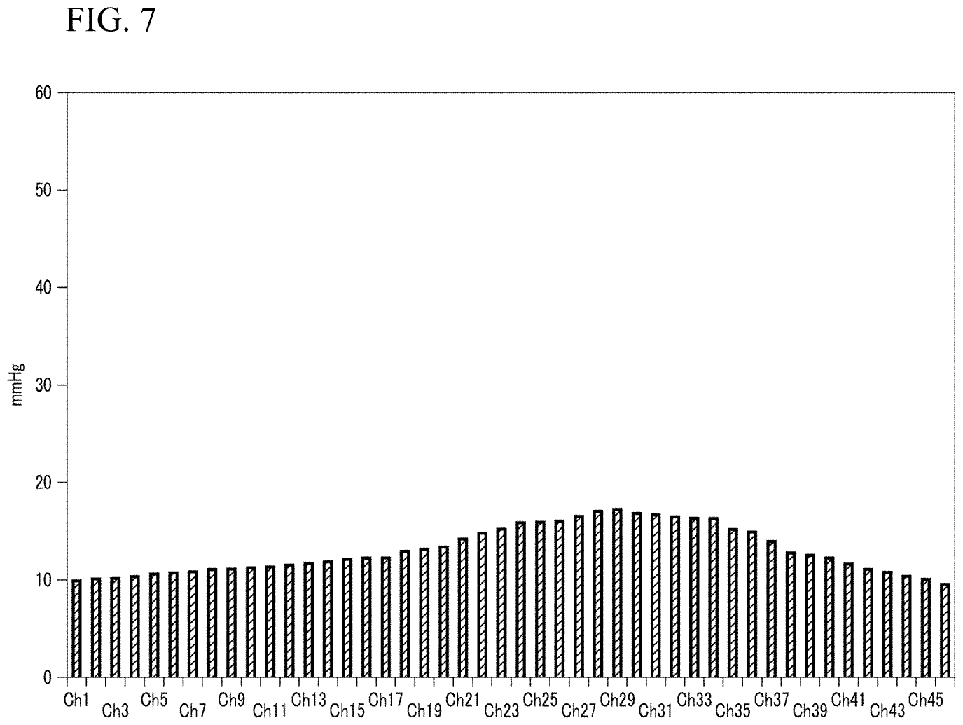

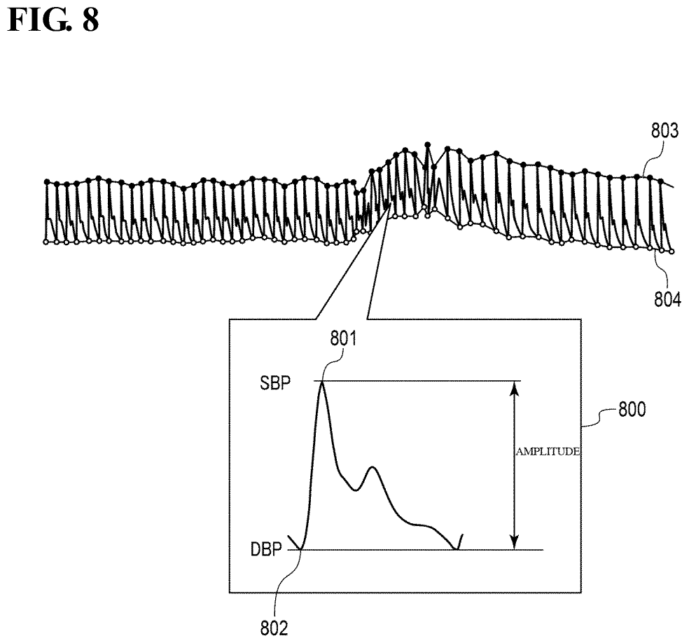

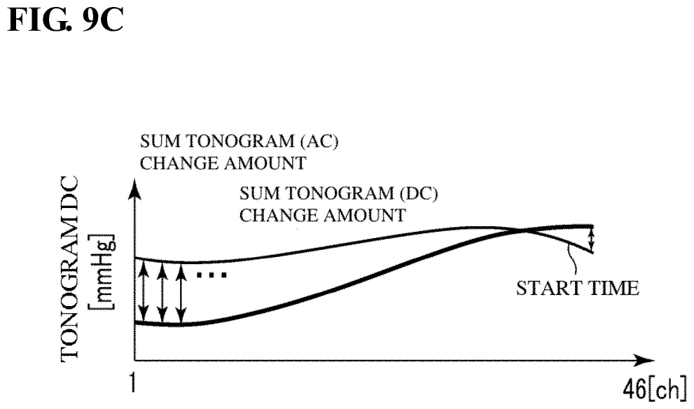

From a distribution of blood pressures per heartbeat in a time series, the feature amount extraction unit 102 extracts a feature amount of the distribution. There are two main types of feature amounts, namely a feature amount extracted from the AC components of a tonogram, and a feature amount extracted from the DC components of a tonogram. Here, “tonogram” refers to the shape of the distribution of calculated feature amounts resulting from the blood pressures for each pressure sensor, with respect to the numbers (e.g., the channel numbers) of multiple pressure sensors. A tonogram is obtained for each sensor array included in the sensor units 201. Also, an AC component of a tonogram corresponds to the difference value between the maximum blood pressure value and the minimum blood pressure value per heartbeat, and a DC component of a tonogram corresponds to the minimum blood pressure per heartbeat. FIG. 6 shows an example of AC components of a tonogram, and FIG. 7 shows an example of DC components of a tonogram. The maximum blood pressure value corresponds to the systolic blood pressure (SBP), and the minimum blood pressure value corresponds to the diastolic blood pressure (DBP). The details of the feature amounts will be described with reference to FIGS. 9A, 9B, and 9C.

The measurement stability determination unit 103 determines whether or not the pressure pulse wave obtained through measurement is stable. For example, the measurement stability determination unit 103 determines whether or not the pulse wave from the blood pressure meter 101 is stable based on a sum of amounts of change in the tonogram (DC) (sum tonogram (DC) change amount) from the prior heartbeat, which is one of the feature amounts extracted by the feature amount extraction unit 102.

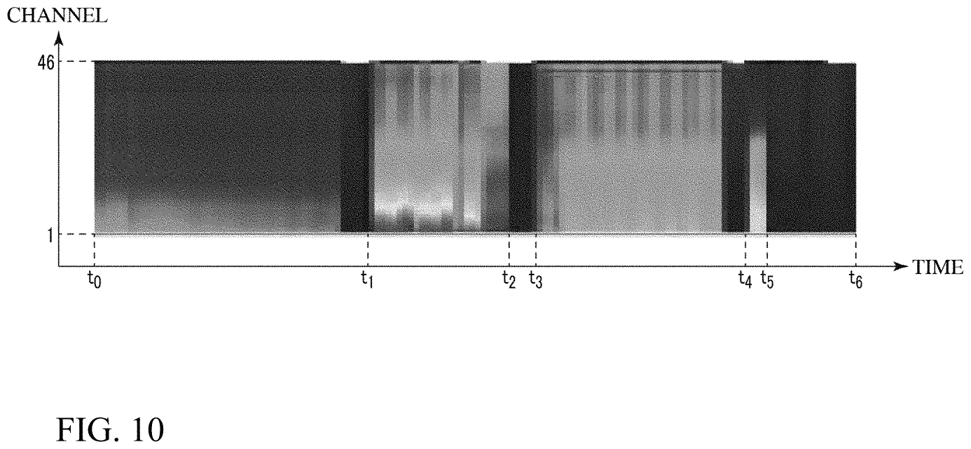

The sum of amounts of change in the tonogram (DC) from the prior heartbeat is obtained by calculating the amount of change in the DC component of the tonogram from the prior heartbeat for each channel, and adding together the change amounts of all channels. The lower the sum tonogram (DC) change amount is in a segment, the more stably the sensor portion 201 can be regarded as being attached to the living body and the more accurate the blood pressure that is acquired can be regarded as being. For example, the measurement stability determination unit 103 of the present embodiment defines a period in which the sum tonogram (DC) change amount is less than or equal to a certain threshold value as a stable segment in which an accurate blood pressure can be stably acquired, and defines a period in which the sum tonogram (DC) change amount is greater than the threshold value as an unstable segment in which an accurate blood pressure cannot be stably acquired. For example, the measurement stability determination unit 103 regards a stable segment as having a high or intermediate degree of reliability, and an unstable segment as having a low degree of reliability. Also, for example, it is possible to employ only blood pressure values detected in stable segments. Specific examples of the stable segment and the unstable segment will be described later with reference to FIG. 10.

The sensor contact state determination unit 104 determines whether or not the state of contact between the sensors (e.g., pressure sensors) used for blood pressure measurement and the measurement site is normal (suitable). For example, the sensor contact state determination unit 104 determines the state of contact based on three feature amounts, namely a tonogram (AC) local maximum value Ch, a tonogram (AC) amplitude difference, and a tonogram (DC) amplitude difference, which are feature amounts extracted by the feature amount extraction unit 102. The tonogram (AC) local maximum value Ch is the channel in which the output value of the AC component of the tonogram reaches a local maximum. Also, the tonogram (AC) amplitude difference is the amplitude difference in the AC components among several adjacent channels in both directions from the channel in which the output value of the AC component of the tonogram reaches a local maximum. Furthermore, the tonogram (DC) amplitude difference is the amplitude difference in the DC components among several channels in both directions from the channel in which the output value of the AC component of the tonogram reaches its local maximum. According to (1) whether or not the tonogram (AC) local maximum value Ch is included in a predetermined range, (2) whether or not the tonogram (AC) amplitude difference is greater than the threshold value, and (3) whether or not the tonogram (DC) amplitude difference is greater than the threshold value, the sensor contact state determination unit 104 determines whether the current state is a tonometry state or a state deviating from the tonometry state. The tonometry state corresponds to a state in which the pressure sensors are suitably arranged with respect to the measurement site in the case of using a blood pressure meter employing tonometry. Regarding (1) above, the tonogram (AC) local maximum value Ch is preferably located near the center (the 23rd channel), and the above-described predetermined range is set as the range of the 15th to 31st channels, for example.

The similarity degree determination unit 105 determines the degree of similarity between the initial state of the pressure pulse wave and the current state of the pressure pulse wave, based on the sum tonogram (AC) change amount and the sum tonogram (DC) change amount, which are feature amounts extracted by the feature amount extraction unit 102. The sum tonogram (AC) change amount is obtained by adding up the change amounts between the output value of each channel at a certain time of the AC component of the tonogram, and an output value (e.g., the average value of each channel in the first minute of measurement) of each channel in the initial state (e.g., during calibration) of the AC component of the tonogram, for all channels. Similarly, the sum tonogram (DC) change amount is obtained by adding up the change amounts between the output value of each channel at a certain time of the DC component of the tonogram and the average value of each channel in the first minute of measurement, for all channels. The time of calibration is a time of converting the output value of the pressure pulse wave into a blood pressure value. The measurement start time is normally the same as the time of calibration. Since the initial state of the tonogram is indicated by the average value of each channel in the first minute of measurement, the similarity degree determination unit 105 can determine how similar the tonogram at a certain time is to the initial tonogram. For example, if the sum tonogram (AC) change amount and the sum tonogram (DC) change amount are both less than respective threshold values (first threshold values), the similarity degree determination unit 105 determines that the degree of similarity is high, and if not, the similarity degree determination unit 105 determines that the degree of similarity is low. In addition, the degree of similarity may also be evaluated as a percentage by associating the values of the sum change amounts with a number of points, and there are various modified examples of the determination result display method.

If it is determined by the similarity degree determination unit 105 that the degree of similarity is low despite the measurement stability determination unit 103 determining that the current segment is a stable segment and the sensor contact state determination unit 104 determining that the current state is a tonometry state, there is a high likelihood that the reference value of the blood pressure has shifted. Examples of this include an orientation change, a change in the position of the wrist, a change in the direction of the wrist, and a change in the attachment state accompanying these changes.

An example was described above in which a blood pressure meter of a tonometry scheme that measures a pressure pulse wave using multiple pressure sensors is used. In the case of using a blood pressure meter that measures the pressure pulse wave using one pressure sensor as well, the measurement stability determination unit 103, the sensor contact state determination unit 104, and the similarity degree determination unit 105 can perform determination processing using a method similar to that described above. In this case, processing for creating a tonogram is not needed. For example, the measurement stability determination unit 103 can determine whether or not the pressure pulse wave is stable based on the amounts of change in the AC components, that is, the differences between the current AC components and the AC components of the prior heartbeat. An AC component corresponds to a value obtained by subtracting the minimum value from the maximum value of the pressure pulse wave waveform of one heartbeat. The sensor contact state determination unit 104 can determine whether or not the state of contact between the pressure sensors and the measurement site is normal based on the output signals of several sensors. The similarity degree determination unit 105 can calculate the degree of similarity between the pressure pulse wave at the measurement start time and the pressure pulse wave at a target measurement time, based on the amounts of change in the AC components and the amounts of change in the DC components. A DC component corresponds to the minimum value of the pressure pulse wave waveform of one heartbeat.

For each measurement segment, the reliability degree calculation unit 107 calculates the degree of reliability of the measurement data from the blood pressure meter 101 based on the determination results of the measurement stability determination unit 103, the sensor contact state determination unit 104, and the similarity degree determination unit 105. For example, with respect to a segment in which the blood pressure data has been determined by the measurement stability determination unit 103 as being in a stable segment, if it is determined by the sensor contact state determination unit 104 that the current state is a tonometry state, the reliability degree calculation unit 107 determines that the degree of reliability is intermediate or higher, and if it is determined the current state is a state deviating from the tonometry state, the reliability degree calculation unit 107 determines that the degree of reliability is low. On the other hand, with respect to a segment in which the blood pressure data has been determined by the measurement stability determination unit 103 as being in an unstable segment, the reliability degree calculation unit 107 determines that the degree of reliability is low. If the sensor contact state determination unit 104 determines that the degree of reliability is intermediate or higher, and furthermore, if the similarity degree determination unit 105 determines that the degree of similarity is high, it is determined that the degree of reliability is high, and if it is determined that the degree of similarity is low, it is determined that the degree of reliability is intermediate. In this manner, the reliability degree calculation unit 107 adds the degree of reliability to each segment for the chronological data of the blood pressure value and stores the degree of reliability in the storage unit 108.

For example, if it is determined by the measurement stability determination unit 103 that the current segment is an unstable segment, the reliability degree calculation unit 107 calculates the degree of reliability as being low without referencing the results of the other determination units. On the other hand, if the current segment is determined as a stable segment by the measurement stability determination unit 103, the sensor contact state determination unit 104 determines whether or not the current state is the tonometry state, but if the current state deviates from the tonometry state, the degree of reliability is calculated as being low without referencing the results of the other determination units.

Also, unlike the description above, the determination results of the determination units may also be indicated by numerical values, and the degree of reliability may also be indicated by a numerical value. The determination results calculated by the determination units 103, 104, and 105 may be subjected to conditional branching, and the degree of reliability may also be displayed as a numerical value. The degree of reliability is high in the case where the current segment is determined as being a stable segment by the measurement stability determination unit 103, the current state is determined as being a tonometry state by the sensor contact state determination unit 104, and the degree of similarity is determined as being high by the similarity degree determination unit 105.

The storage unit 108 stores the blood pressure data from the blood pressure meter 101 and the degree of reliability thereof in association with each other. For example, the storage unit 108 may also store the blood pressure data and the degree of reliability thereof in association with each other for each user. The storage unit 108 records the blood pressure data from the blood pressure meter 101 along with the degree of reliability.

The blood pressure meter 101 will be described next with reference to FIG. 2.

The blood pressure meter 101 includes: a sensor unit 201, a pressing portion 202, a control unit 203, a storage unit 204, an operation unit 205, and an output unit 206. The sensor unit 201 continuously detects the pressure pulse wave over time. For example, the sensor units 201 detect the pressure pulse wave for each heartbeat. The sensor units 201 include sensors that detect pressure, are arranged on the side of the wrist corresponding to the palm of the hand as shown in FIG. 3, and are normally arranged in parallel in two rows in the extension direction of the arm as shown in FIG. 3. In each row of the sensor array including the multiple sensors, multiple (e.g., 46) sensors are arranged intersecting (approximately perpendicular to) the extension direction of the arm. The pressing portion 202 is composed of a pump, a valve, a pressure sensor, and an air bag, and can increase the sensitivity of the sensors by pressing the sensors of the sensor units 201 to the wrist with a suitable pressure due to an air bag inflating. Air is inserted into the air bag through the pump and the valve, the pressure sensor detects the pressure inside of the air bag, and the control unit 203 performs monitoring and controlling to perform adjustment to a suitable pressure. The control unit 203 performs overall control of the blood pressure meter 101, receives chronological data of the pulse wave from the sensor units 201, converts the data into chronological data of the blood pressure values, and stores the result as blood pressure data in the storage unit 204. The storage unit 204 stores the blood pressure data and transfers desired data in response to a request from the control unit 203. The operation unit 205 receives input from a user or the like from a keyboard, a mouse, a microphone, or the like, and receives an instruction from an external server or the like through a wire or wirelessly. The output unit 206 receives the blood pressure data stored in the storage unit 204 via the control unit 203 and transmits the blood pressure data to the outside of the blood pressure meter 101.

The blood pressure measurement apparatus 100 is arranged on the side of the wrist corresponding to the palm of the hand, as shown in FIGS. 3 and 4, and the sensor units 201 of the blood pressure meter 101 are arranged so as to be located on the radial artery RA. As indicated by the arrow in FIG. 4, the pressing portion 202 presses the sensor units 201 to the wrist W and presses flat the radial artery RA. Note that the blood pressure measurement apparatus 100 is ring-shaped, wraps like a bracelet around the wrist or the like, and measures the blood pressure, although this is not shown in FIGS. 3 and 4.

Next, the sensor units 201 of the blood pressure measurement apparatus 100 will be described with reference to FIG. 5. FIG. 5 shows a surface on the side of the sensor units 201 that comes into contact with the wrist W. As shown in FIG. 5, the sensor units 201 include one or more (in this example, two) sensor arrays, and each sensor array includes multiple sensors aligned in the direction B. The direction B is a direction that intersects a direction A in which the radial artery extends in a state in which the blood pressure measurement apparatus 100 is attached to the measurement subject. For example, the direction A and the direction B may also be perpendicular. For example, 46 sensors (referred to as 46 channels) are arranged in one row. Note that here, the sensors are provided with channel numbers. Also, the arrangement of the sensors is not limited to the example shown in FIG. 5.

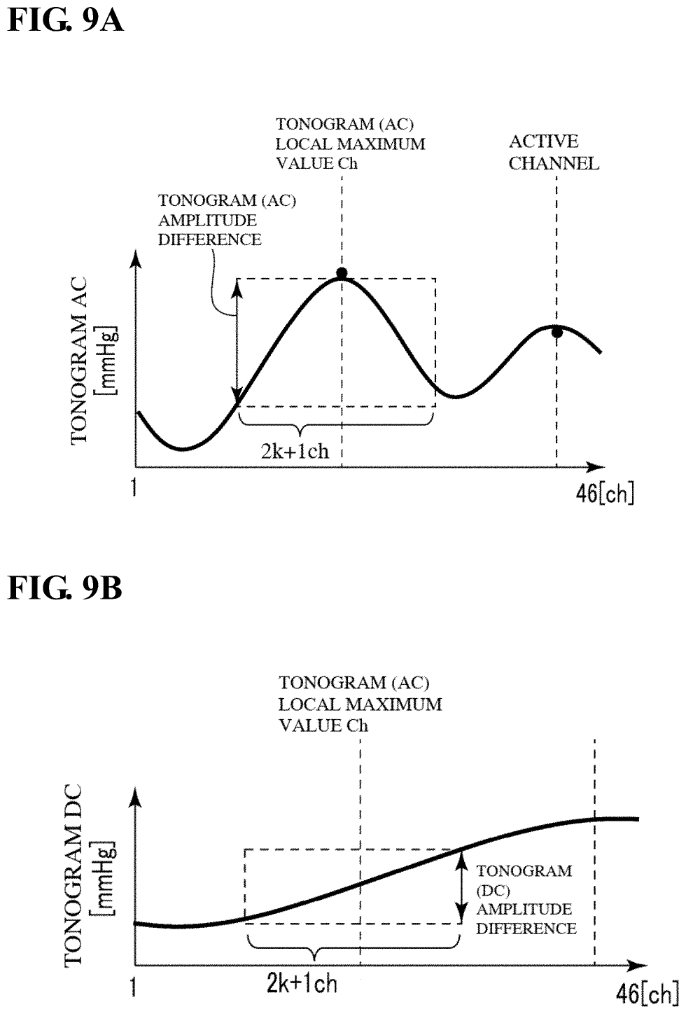

The sensors generate pressure data by measuring the pressure. Piezoelectric elements that convert pressure into electrical signals can be used as the sensors. A pressure waveform as shown in FIG. 8 is obtained as the pressure data. The result of measuring the pressure pulse wave is generated based on the pressure data output from one sensor (active channel) selected adaptively from among the sensors. The maximum value in the waveform of a pressure pulse wave of one heartbeat corresponds to the SBP, and the minimum value in the waveform of a pressure pulse wave of one heartbeat corresponds to the DBP. The blood pressure data can include the result of measuring the pressure pulse wave and the pressure data output from each of the sensors. Note that the result of measuring the pulse wave may also be generated based on the pressure data by the control unit 203 including the information processing unit in the blood pressure measurement apparatus 100, without being generated in the blood pressure meter 101.

Next, chronological data calculated based on the pressure pulse wave measured by the blood pressure meter 101 will be described with reference to FIG. 8. FIG. 8 shows choronological data of the blood pressure calculated based on the pressure pulse wave when the pressure pulse wave for each heartbeat is measured. Also, FIG. 8 shows a waveform of blood pressure obtained based on one of the pressure pulse waves. The blood pressure obtained based on the pressure pulse wave is detected for each heartbeat as a waveform such as that shown in FIG. 8, and the blood pressure obtained based on the pressure pulse waves is continuously detected. A waveform 800 shown in FIG. 8 is a blood pressure waveform obtained based on the pressure pulse wave of one heartbeat, an output value indicated by reference number 801 corresponds to the SBP, and an output value indicated by reference number 802 corresponds to the DBP. As indicated by the time series of the blood pressure corresponding to the pressure pulse wave of FIG. 8, the SBP 803 and the DBP 804 of the blood pressure pulse wave fluctuate for each heartbeat.

A feature amount extracted by the feature amount extraction unit 102 will be described with reference to FIGS. 9A, 9B, and 9C. FIGS. 9A, 9B, and 9C show feature amounts extracted by the feature amount extraction unit 102, taking the example of graphs of AC components and DC components of an exemplary tonogram.

The sum of the amounts of change in the tonogram (DC) between the prior heartbeat and the current heartbeat, which is a feature amount used by the measurement stability determination unit 103, is obtained by calculating the amount of change in the DC component of the tonogram between the prior heartbeat and the current heartbeat for each channel, and adding up the change amounts of all channels. There are three types of feature amounts used by the sensor contact state determination unit 104, which are the tonogram (AC) local maximum value Ch, the tonogram (DC) amplitude difference, and the tonogram (AC) amplitude difference. The tonogram (AC) local maximum value Ch is the channel in which the output value of the AC component of the tonogram is at a local maximum, as shown in FIG. 9A. The tonogram (DC) amplitude difference is the amplitude difference in the DC components in the tonogram corresponding to k (e.g., k=10) adjacent channels in both directions from the channel in which the AC component of the tonogram reaches a local maximum, as shown in FIG. 9B. The tonogram (AC) amplitude difference is the amplitude difference in the AC components in the tonogram corresponding to k adjacent channels in both directions from the channel in which the AC component of the tonogram reaches its local maximum, as shown in FIG. 9A.

There are two types of feature amounts used by the similarity degree determination unit 105, which are the sum tonogram (AC) change amount, and the sum tonogram (DC) change amount. The sum tonogram (AC) change amount is obtained by calculating the amounts of change between the output value of the channels at a certain time t of the AC components of the tonogram, and the initial output values of the channels, and adding up the change amounts of all of the channels. Here, the initial output values of the channels are, for example, the average values of the output values of each channel in the first minute of measurement. Also, the sum tonogram (DC) change amount is obtained by replacing the AC components with the DC components in the sum tonogram (AC) change amount.

Next, a stable segment and an unstable segment will be described with reference to FIG. 10. In FIG. 10, the horizontal axis indicates time, the vertical axis indicates the channel number of the sensor array, and the magnitudes of the output values of the sensors are indicated through shading. From time t₀ to t₁ and from time t₅ to t₆ in FIG. 10, the brighter the color is, the greater the output value is, and the darker the color is, the smaller the output value is. From time t₁ to t₂ and from time t₃ to t₄ in FIG. 10, the darker the color is, the greater the output value is. That is, it can be understood that from time t₅ to t₆, the output values of the sensors are approximately smaller than those from time t₀ to t₁. Also, from time t₀ to t₁, the output values from around channels 1 to 10 are greater than the output values of channels 10 and onward. Also, from time t₀ to t₁ as well, the output values from around channels 1 to less than 10 are greater than those of the other channels up to channel 46. In the case of FIG. 10, time t₀ to t₁ corresponds to a stable segment, time t₁ to t₂ corresponds to an unstable segment, time t₃ to t₄ corresponds to an unstable segment, and time t₅ to t₆ corresponds to a stable segment.

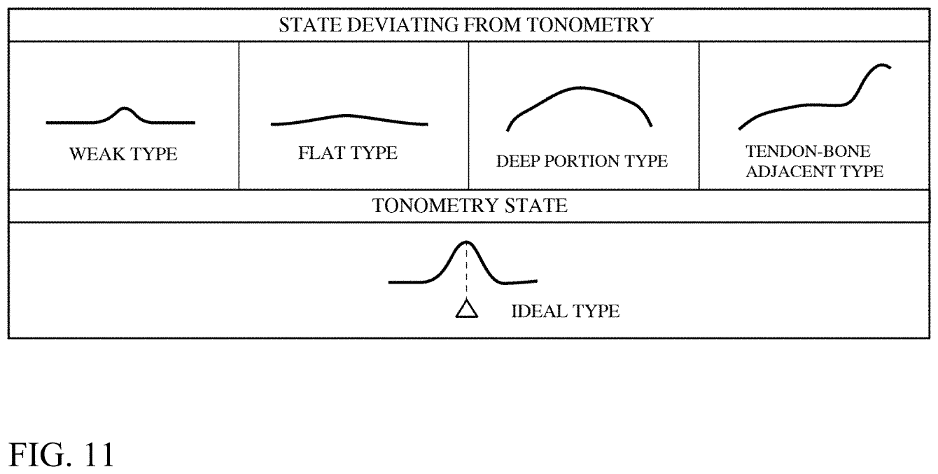

Next, examples of typical tonograms in a tonometry state and a state deviating from the tonometry state will be described with reference to FIG. 11. In the four tonograms shown in the upper portion of FIG. 11, the horizontal direction (horizontal axis) indicates the channel numbers of the sensors, and the vertical direction (vertical axis) indicates the output values (e.g., blood pressure values) of the sensors. The four upper portions indicate states deviating from the tonometry state, and the one lower portion indicates the tonometry state. The two left upper portions typically indicate cases in which the pulse is weak, the third upper portion from the left indicates a case in which there is a high likelihood that the pulse is located in a deep portion, or the sensors are arranged toward the elbow, and the example of the rightmost upper portion is a case in which a tendon has a significant influence, and for example, the wrist is thin. The tonometry state is characterized in that the output value of the central portion of the channels is great (there is one local maximum value and the amplitude thereof is greater than a certain value), and the output values slope more gently with left-right symmetry toward the channels at both ends. The measurement stability determination unit 103 performs determination of the tonometry state.

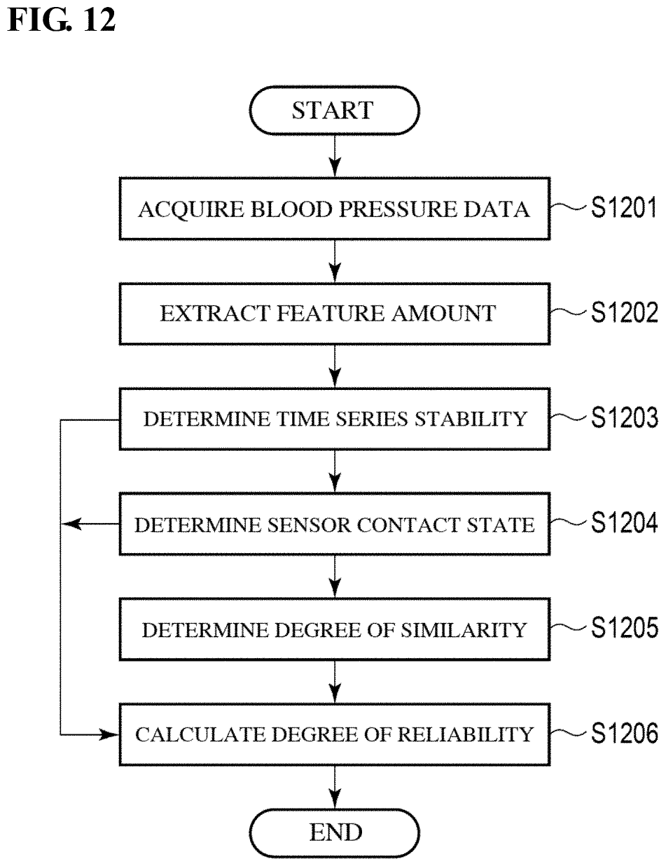

Next, an example of operations of the blood pressure measurement apparatus 100 will be described with reference to FIG. 12. FIG. 12 is a flowchart showing a typical example of operations of the blood pressure measurement apparatus 100. The blood pressure meter 101 acquires the chronological data from the living body and transfers it to the feature amount extraction unit 102 (step S1201). The blood pressure meter 101 transfers the chronological data to the storage unit 108 and the storage unit 108 sequentially records the chronological data of the blood pressure values.

In step S1202, the feature amount extraction unit 102 extracts the feature amounts required by the measurement stability determination unit 103, the sensor contact state determination unit 104, and the similarity degree determination unit 105, and transfers the feature amounts corresponding to the respective determination units.

In step S1203, if the sum of amounts of change in the tonogram (DC) from the prior heartbeat, which was received from the feature amount extraction unit 102, is less than or equal to a threshold value, the measurement stability determination unit 103 determines that the current segment is a stable segment, and if it is another value, the measurement stability determination unit 103 determines that the current segment is an unstable segment (step S1203). If the measurement stability determination unit 103 determines that the current segment is a stable segment, the processing advances to step S1204. If the measurement stability determination unit 103 determines that the current segment is an unstable segment, the processing advances to step S1206 and the reliability degree calculation unit 107 determines that “the degree of reliability is low”.

In step S1204, the sensor contact state determination unit 104 determines whether or not the current state is a tonometry state based on (condition 1) whether or not the tonogram (AC) local maximum value Ch, which was received from the feature amount extraction unit 102, is included in a predetermined range, (condition 2) whether or not the tonogram (AC) amplitude difference is greater than a threshold value, and (condition 3) whether or not the tonogram (DC) amplitude difference is greater than a threshold value. For example, if (condition 1), (condition 2), and (condition 3) are all satisfied, the sensor contact state determination unit 104 determines that the current state is a tonometry state and the processing advances to step S1205. If even one of (condition 1), (condition 2), and (condition 3) is not satisfied, the sensor contact state determination unit 104 determines that the current state is a state deviating from the tonometry state, the processing advances to step S1206, and the reliability degree calculation unit 107 determines that “the degree of reliability is low”.

In step S1205, the similarity degree determination unit 105 determines the degree of reliability of the tonogram based on the two feature amounts, namely the sum tonogram (AC) change amount and the sum tonogram (DC) change amount, which were received from the feature amount extraction unit 102. For example, if the sum tonogram (AC) change amount and the sum tonogram (DC) change amount are both smaller than respective threshold values (first threshold values), the similarity degree determination unit 105 determines that the degree of similarity is high, and the reliability degree calculation unit 107 determines that the degree of reliability is high (step S1206). On the other hand, if at least one of the sum tonogram (AC) change amount and the sum tonogram (DC) change amount is greater than or equal to the threshold value, the similarity degree determination unit 105 determines that the degree of similarity is low, and the reliability degree calculation unit 107 determines that the degree of reliability is intermediate (step S1206).

Note that in this example, the measurement stability determination unit 103, the sensor contact state determination unit 104, and the similarity degree determination unit sequentially perform determination processing and transfer the determination results to the reliability degree calculation unit 107, but alternatively, the measurement stability determination unit 103, the sensor contact state determination unit 104, and the similarity degree determination unit 105 may also perform determination processing in parallel, and the reliability degree calculation unit 107 may suitably perform conditional branching on the determination results to determine the reliability degree.

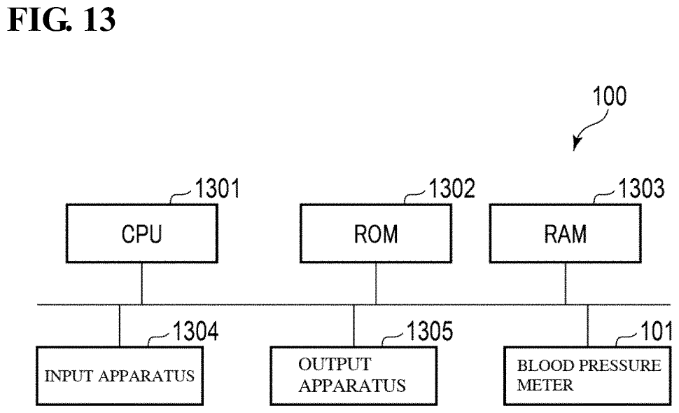

Next, an example of a hardware configuration of the blood pressure measurement apparatus 100 will be described with reference to FIG. 13.

The blood pressure measurement apparatus 100 includes a CPU 1301, a ROM 1302, a RAM 1303, an input apparatus 1304, an output apparatus 1305, and a blood pressure meter 101, and these elements are connected to each other via a bus system 1306. The above-described functions of the blood pressure measurement apparatus 100 can be realized by the CPU 1301 reading out a program stored in a computer-readable recording medium (ROM 1302) and executing the read-out program. The RAM 1303 is used by the CPU 1301 as a work memory. In addition, an auxiliary storage apparatus (not shown) such as a hard disk drive (HDD) or a solid-state drive (SSD) may also be included, used as the storage unit 108, and further store a program. For example, the input apparatus 1304 includes a keyboard, a mouse, and a microphone, and receives operations from a user. For example, the input apparatus 1304 includes an operation button for causing the blood pressure meter 101 to start measurement, an operation button for performing calibration, and an operation button for starting or stopping communication. For example, the output apparatus 1305 includes a display apparatus such as a liquid crystal display apparatus, and a speaker. The blood pressure meter 101 performs transmission and reception of signals with another computer using a communication apparatus, for example, and receives measurement data from a blood pressure measurement apparatus, for example. The communication apparatus often uses a communication scheme according to which data can be mutually exchanged at a short distance, and for example, uses a near-field wireless communication scheme, specific examples of which include communication schemes such as Bluetooth (registered trademark), TransferJet (registered trademark), ZigBee (registered trademark), and IrDA (registered trademark).

Also, a program for executing the operations performed by the above-described feature amount extraction unit 102, measurement stability determination unit 103, sensor contact state determination unit 104, similarity degree determination unit 105, and reliability degree calculation unit 107 may also be stored in the above-described ROM 1302 or the auxiliary storage apparatus, and the program may be executed by the CPU 1301. Alternatively, the program may also be stored in a server or the like separate from the blood pressure measurement apparatus 100 and the CPU of the server or the like may execute the program. In this case, the degree of reliability can be obtained by transmitting the chronological data of the pressure pulse wave (or the chronological data of the blood pressure values) measured by the blood pressure meter 101 to the server and performing processing in the server. In this case, there is a possibility that the processing speed will increase since the processing is performed in the server. Furthermore, the apparatus portions of the feature amount extraction unit 102, the measurement stability determination unit 103, the sensor contact state determination unit 104, the similarity degree determination unit 105, and the reliability degree calculation unit 107 are removed from the blood pressure measurement apparatus 100, and therefore the size of the blood pressure measurement apparatus 100 is smaller, and the sensors can be easily arranged at a position at which measurement can be performed accurately. In this case, the burden on the user can be reduced, and accurate blood pressure measurement can be easily performed.

According to the blood pressure measurement apparatus of the above-described embodiment, the degree of reliability for the measured blood pressure value can be evaluated each instance of measurement (e.g., each heartbeat), and thus the degree of reliability for the measured blood pressure value can be evaluated according to the measurement target. Also, if it is determined that the pressure pulse wave is stable and it is determined that the tonogram is in a tonometry state, and furthermore, if it is determined that the degree of similarity is greater than a first threshold value, the calculation unit calculates the degree of reliability in that segment as being the highest, whereby it is determined that the stability of the minimum blood pressure value is high, the tonogram is in the tonometry state, and the degree of similarity with the measurement start time of the tonogram is high. In this case, it can be understood that blood pressure data in the best conditions has been obtained. As a result, the fact that the there is little shifting in the sensors from the radial artery and the pressure pulse wave is reliably received can be incorporated into the degree of reliability, and more ideal continuous blood pressure data for each heartbeat can be obtained.

Since there are individual differences in the positional relationship between the radial artery and the radius and tendon, a person who has thick subcutaneous tissue on the radial artery, a person whose radial artery, radius, and tendon are near each other, or the like performs measurement in a state in which the degree of reliability is low. However, by additionally incorporating whether or not the pressure pulse wave is stable, and the degree of similarity between the tonogram at the measurement start time and the tonogram at a desired measurement time into the conditions for determining the degree of reliability of the chronological data of the blood pressure values, it is possible to evaluate a degree of reliability of the blood pressure values that is more suitable for the actual measurement environment. Also, due to incorporating the stability of the pressure pulse wave and the degree of similarity into the determination conditions in this manner, the degree of reliability can change according to the measurement conditions in the same living body.

Furthermore, since the conversion from the pressure value to the blood pressure value is calculated based on the tonogram information at the time of calibration, the reliability of the blood pressure values is not completely evaluated merely by evaluating the degree of reliability using the tonogram information for each heartbeat. However, by incorporating the degree of similarity into the determination conditions in this manner, the degree of similarity to the tonogram at the time of calibration, which is usually performed each time measurement is started, is evaluated, and thus it is possible to obtain the degree of reliability regarding whether or not the blood pressure value is correct. For example, even if the degree of reliability increases due to a determination condition other than the degree of similarity during measurement, the correct blood pressure value will not be calculated if the tonogram is not similar to that at the time of calibration, and therefore it is possible to perform a calculation for reducing the degree of reliability, and thus it is understood that a degree of reliability obtained with consideration given to the degree of similarity of the blood pressure measurement apparatus of the present embodiment is more accurate.

The apparatus of the present invention can be realized also by a computer or a program, and the program can be recorded in a recording medium and can be provided via a network.

Also, the above-described apparatuses and the apparatus portions thereof can be implemented by any hardware configuration or combination configuration of hardware resources and software. A program for causing a computer to perform functions of the apparatuses by being installed in a computer in advance from a network or a computer-readable recording medium and being executed by a processor of the computer is used as the software of the combination configuration.

Note that the present invention is not limited to the above-described embodiment as-is, and can be realized with modifications to the constituent elements without departing from the gist in the implementation stage. Also, various aspects of the invention can be formed through suitable combinations of the multiple constituent elements disclosed in the above-described embodiment. For example, several constituent elements may also be removed from all of the constituent elements shown in the embodiment. Furthermore, the constituent elements of different embodiments may also be combined as appropriate.

Also, a portion or all of the above-described embodiment can be described as in the following supplementary notes, but there is no limitation to the following description.

### Supplementary Note 1

A blood pressure measurement apparatus including a hardware processor and a memory, wherein

the hardware processor is configured to:
obtain blood pressure data including a blood pressure value for each heartbeat by detecting a pressure pulse wave using one or more sensors,
extract one or more feature amounts of the blood pressure data, and
calculate a degree of reliability indicating how accurately the blood pressure data indicates blood pressure values, based on the feature amount, and

the memory includes a storage unit configured to store the degree of reliability and the blood pressure data.

### Supplementary Note 2

A blood pressure measurement method comprising:

obtaining blood pressure data including a blood pressure value for each heartbeat by detecting a pressure pulse wave using one or more sensors, using at least one hardware processor;

extracting one or more feature amounts of the blood pressure data using at least one hardware processor; and

calculating a degree of reliability indicating how accurately the blood pressure data indicates blood pressure values, based on the feature amount, using at least one hardware processor.

1. A blood pressure measurement apparatus comprising:
a blood pressure meter configured to obtain chronological blood pressure data including a blood pressure value for each heartbeat by detecting a pressure pulse wave using one or more sensors; and
a processor programmed to:
extract one or more feature amounts of the chronological blood pressure data; and
calculate, for each time segment determined using the feature amount, a degree of reliability indicating how accurately the chronological blood pressure data indicates blood pressure values, based on the feature amount.

2. The blood pressure measurement apparatus according to claim 1, wherein the processor is programmed to:
extract at least one feature amount among a stability feature amount indicating whether or not the pressure pulse wave is stable, a sensor contact state feature amount indicating whether or not a state of contact between the one or more sensors and a measurement site is normal, and a similarity degree feature amount indicating a degree of similarity between the pressure pulse wave at a measurement start time and the pressure pulse wave at a desired measurement time, and
calculate the degree of reliability based on the at least one feature amount.

3. The blood pressure measurement apparatus according to claim 2, wherein upon determining that the pressure pulse wave is stable and it is determined that the state of contact is normal, and upon determining that the degree of similarity is greater than a threshold value, the processor is programmed to set the degree of reliability for that time segment as being high.

4. A blood pressure measurement method comprising:
obtaining chronological blood pressure data including a blood pressure value for each heartbeat by detecting a pressure pulse wave using one or more sensors,
extracting one or more feature amounts of the chronological blood pressure data, and
calculating, for each time segment determined using the feature amount, a degree of reliability indicating how accurately the chronological blood pressure data indicates blood pressure values, based on the feature amount.

5. The blood pressure measurement apparatus according to claim 1, wherein the processor is programmed to:
extract at least one feature amount among:
a stability feature amount indicating whether or not the pressure pulse wave is stable, the stability feature amount being calculated based on a sum change amount obtained by calculating, for each channel, an amount of change in a DC component of a tonogram from a prior heartbeat, and adding together the amounts of change of all channels,
a sensor contact state feature amount indicating whether or not a state of contact between the one or more sensors and a measurement site is normal, the sensor contact state being calculated based on three feature amounts, namely a channel in which an output value of an AC component of a tonogram reaches a local maximum, an amplitude difference in AC components among several adjacent channels in both directions from the channel in which the output value of the AC component of the tonogram reaches a local maximum, and an amplitude difference in DC components among several adjacent channels in both directions from the channel in which the output value of the AC computer of the tonogram reaches a local maximum, and
a similarity degree feature amount indicating a degree of similarity between the pressure pulse wave at a measurement start time and the pressure pulse wave at a desired measurement time, and
calculate the degree of reliability based on the at least one feature amount.

6. A non-transitory computer readable storage medium storing instructions causing a computer to function as the blood pressure measurement apparatus according to claim 1.

7. A non-transitory computer readable storage medium storing instructions causing a computer to function as the blood pressure measurement apparatus according to claim 2.

8. A non-transitory computer readable storage medium storing instructions causing a computer to function as the blood pressure measurement apparatus according to claim 3.

9. A non-transitory computer readable storage medium storing instructions causing a computer to function as the blood pressure measurement apparatus according to claim 5.
